(19) United States
(12) Patent Application Publication
(10) Pub. No.: US 2026/0232292 A1
(43) Pub. Date: Aug. 13, 2026

(54) System, Device and Method for Medical Imaging

(71) Applicant: IMEC VZW, Leuven (BE)

(72) Inventor: Patrick Gross, Crawley Down (GB)

(21) Appl. No.: 19/536,911

(22) Filed: Feb. 11, 2026

(30) Foreign Application Priority Data
Feb. 12, 2025 (EP) 25157332.5

(51) Int. Cl. A61B 8/14; A61B 8/00

(52) U.S. Cl. CPC A61B 8/14; A61B 8/463; A61B 8/5207

(57) ABSTRACT

An imaging system for medical imaging is provided. The imaging system includes at least one training device, and at least one imaging device operably coupled to the at least one training device. The at least one imaging device is configured to be attached to the skin of a person at a predefined location. The at least one imaging device, in combination with the at least one training device, is configured to generate a first set of images corresponding to the predefined location, whereby the at least one training device is configured to generate training data corresponding to the predefined location based on the first set of images. Furthermore, the at least one imaging device is further configured to generate a second set of images corresponding to the predefined location based on the training data.

### CROSS-REFERENCE TO RELATED APPLICATION(S)

The present application is a non-provisional patent application claiming priority to European Patent Application No. 25157332.5, filed February 12, 2025, the contents of which are hereby incorporated by reference.

### FIELD OF THE DISCLOSURE

The disclosure relates to medical imaging and/or targeted drug delivery, particularly to medical therapeutic imaging and/or targeted therapeutic drug delivery, in a (e.g., largely) autonomous manner.

### BACKGROUND

Generally, when delivering drugs intravascularly, e.g., either intravenously or via a catheter, at least a (e.g., significant) proportion circulates systemically, potentially causing toxicity. In addition, depending on the molecular size and biochemical properties of the drugs, the drugs may have difficulty crossing from the vasculature into the target tissue, e.g., crossing the blood-brain-barrier.

Both these challenges may be addressed using bubble agents that oscillate at their resonant frequency using ultrasound. The oscillations may open and make the vasculature more permeable and/or release drugs by bursting bubbles containing them. This may be done in a locally targeted manner using ultrasound.

For example, the document US 2024/0131366 A1 discloses a Low-Intensity Non-Focused Ultrasound (LINFU) method and device for creating a wide area of stable cavitation through resonance of intravenously introduced microbubbles inside the (e.g., entire) pancreas or another organ-sized area of the body.

However, applying a targeted delivery regime over an extended period of time, using conventional technology, such as the LINFU method of US 2024/0131366 A1, or using non-trainable devices (e.g., based on operator or trained professionals) may not be feasible. Additionally, the automation of such heavy and rigid devices is not practical, because the user of such devices, e.g., a patient, may have to remain stationary for a long time and any motion may degrade the acoustic coupling.

### SUMMARY

The disclosure provides a system, a device, and a method for medical therapeutic imaging and/or targeted drug delivery in a reliable autonomous manner.

The features of the first independent claim for the system, the features of the second independent claim for the device, and the features of the third independent claim for the method are provided. The dependent claims contain further developments.

According to a first example embodiment of the disclosure, an imaging system for medical imaging is provided. The imaging system includes at least one training device, and at least one imaging device operably coupled to the at least one training device. The at least one imaging device is configured to attach to the skin of a person at a predefined location.

The at least one imaging device, in combination with the at least one training device, is configured to generate a first set of images corresponding to the predefined location. The at least one training device is configured to generate training data corresponding to the predefined location based on the first set of images.

Furthermore, the at least one imaging device is further configured to (e.g., exclusively) generate a second set of images corresponding to the predefined location based on the training data.

The term imaging may be understood as acoustic imaging performed by the at least one imaging device at the predefined location, e.g., corresponding to a target organ. Additionally or alternatively, the term imaging may be understood as spatial and/or temporal sensing using sensing data, either unreconstructed or raw sensing data or processed sensing data, generated by the at least one imaging device.

The at least one imaging device, in combination with the at least one training device, may generate a first set of sensing data, e.g., from spatial and/or temporal sensing at the predefined location, corresponding to a target organ or corresponding to the predefined location. The at least one training device may thereby generate training data corresponding to the predefined location based on the first set of sensing data.

Afterwards, the at least one imaging device may (e.g., exclusively) generate, e.g., from a remote location corresponding to the at least one training device, a second set of sensing data corresponding to the predefined location based on the training data. The relative location of an example anatomy may be deduced, and targeted drug delivery, such as using bubble agents, may be performed in a (e.g., largely) autonomous manner.

The at least one training device includes an ultrasound imaging device, an x-ray imaging device, a computed tomography (CT) device, a magnetic resonance imaging (MRI) device, a positron emission tomography (PET) device, or a combination thereof.

The first set of images and the second set of images are ultrasound images, e.g., Doppler ultrasound imaging and/or sensing, brightness mode (B-Mode) imaging and/or sensing, or harmonic imaging and/or sensing.

The training data includes instructions for the operating frequency of the at least one imaging device, and/or focus of the second set of images corresponding to the predefined location, and/or depth of the second set of images corresponding to the predefined location, and/or attachment anomalies between the at least one imaging device and the skin. In some example embodiments, utilizing such training data could improve the reliability of the imaging system.

The training data further includes parameters for a focused ultrasound based on the first set of images, and the at least one imaging device is further configured to perform the focused ultrasound at the predefined location during the generation of the second set of images based on the training data. A non-invasive and non-radiative way (e.g., means) for targeting tissue, e.g., for high intensity deeper tissue treatment, is facilitated.

The at least one imaging device is configured to transmit the second set of images to the at least one training device, optionally wirelessly.

The at least one training device is configured to update the training data based on the second set of images and further configured to transmit the updated training data to the at least one imaging device, optionally wirelessly.

The at least one training device is configured to control at least one parameter of the at least one imaging device, optionally wirelessly.

The at least one parameter includes the operating frequency of the at least one imaging device, focus of the second set of images corresponding to the predefined location, and/or depth of the second set of images corresponding to the predefined location.

In various systems, devices, and methods described herein, the reliability of the at least one imaging device may be improved.

The imaging system further includes a storage, optionally a cloud storage, configured to store the first set of images, the second set of images, and/or the training data.

According to a second example embodiment of the disclosure, an imaging device for medical imaging is provided. The imaging device includes a flexible substrate configured to attach to the skin of a person at a predefined location. The imaging device further includes a processor configured to process training data corresponding to the predefined location based on a first set of images generated in combination with at least one training device according to the first example embodiment of the disclosure.

Moreover, the imaging device includes at least one imaging module configured to generate a second set of images corresponding to the predefined location based on the training data. The processor and the at least one imaging module are arranged on the flexible substrate.

The flexible substrate is configured to attach to the skin of the person at the predefined location mechanically or via an adhesive.

The at least one imaging module is a transducer, optionally an ultrasound transducer.

The imaging device further includes a transceiver configured to receive the training data from the at least one training device, optionally wirelessly. Additionally or alternatively, the transceiver is configured to transmit the second set of images to the at least one training device, optionally wirelessly.

According to a third example embodiment of the disclosure, an imaging method for medical imaging is provided. The method includes generating, by at least one imaging device attached to the skin of a person at a predefined location, in combination with at least one training device, a first set of images corresponding to the predefined location. The method further includes generating, by the at least one training device, training data corresponding to the predefined location based on the first set of images.

Moreover, the method includes generating, (e.g., exclusively) by the at least one imaging device, a second set of images corresponding to the predefined location based on the training data.

The method according to the third example embodiment corresponds to the system according to the first example embodiment and its implementation forms. The imaging device according to the second example embodiment corresponds to the imaging device according to the first example embodiment and its implementation forms.

### BRIEF DESCRIPTION OF THE FIGURES

The above, as well as additional, features will be better understood through the following illustrative and non-limiting detailed description of example embodiments, with reference to the appended drawings.

Example embodiments of the disclosure are now further provided with respect to the drawings by way of example only, and not for limitation.

FIG. 1 shows a first example embodiment of the imaging system according to the disclosure.

FIG. 2 shows a second example embodiment of the imaging system according to the disclosure.

FIG. 3 shows a third example embodiment of the imaging system according to the disclosure.

FIG. 4 shows an example embodiment of the imaging device according to the disclosure.

FIG. 5A shows an example training phase of the imaging system.

FIG. 5B shows an example imaging and/or delivery phase of the imaging system.

FIG. 6 shows an example embodiment of the method according to the disclosure.

All the figures are schematic, not necessarily to scale, and generally show parts to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Reference will now be made in detail to the example embodiments of the present disclosure, examples of which are provided in the accompanying drawings. However, the following example embodiments of the present disclosure may be (e.g., variously) modified and the range of the present disclosure is not limited by the following example embodiments.

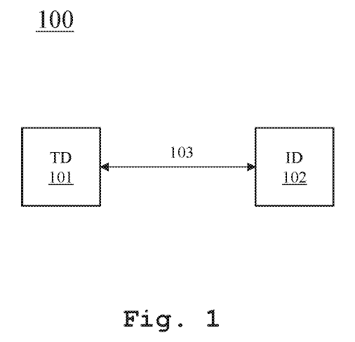

FIG. 1 shows a first example embodiment of an imaging system 100 according to the disclosure. The term imaging may be understood as acoustic imaging performed at a predefined location, e.g., corresponding to a target organ. Additionally or alternatively, the term imaging may be understood as spatial and/or temporal sensing at the predefined location, e.g., corresponding to a target organ.

The imaging system 100 may include a training device (TD) 101 and an imaging or sensing device (ID) 102. The imaging device 102 may be coupled to the training device 101 via a link 103, which may be a wired link or a wireless link. For example, the imaging device 102 may be attached to the skin of a user, e.g., a patient, at a predefined location, e.g., corresponding to a target organ or tissue.

During a first phase of operation or the training phase, the imaging device 102, in combination with the training device 101, may generate a first set of images and/or sensing data corresponding to the predefined location and/or target organ or tissue, and the training device 101 may generate training data corresponding to the predefined location and/or target organ or tissue based on the first set of images and/or sensing data.

During a second phase of operation or the delivery phase, the imaging device 102 may (e.g., exclusively) generate a second set of images and/or sensing data corresponding to the predefined location and/or target organ or tissue based on the training data and/or may perform a (e.g., specific) task based on the second set of images and/or sensing data, such as from a location remote to the training device 101.

For example, the training device 101 may include an ultrasound imaging device, an x-ray imaging device, a CT device, an MRI device, a PET device, or a combination thereof, during the training phase to facilitate the training of the imaging device 102.

For example, the images may correspond to ultrasound images of a target organ, e.g., the pancreas, which may be monitored, e.g., for detecting bubble agent resonance. Additionally or alternatively, the sensing data may include unreconstructed or raw sensing data or processed sensing data, from which ultrasound images may be realized, e.g., via the training device 101, of a target organ, e.g., the pancreas. Thus, the images reconstructed from the raw sensing data may also be monitored, e.g., for detecting bubble agent resonance.

For example, during the training phase, the training device 101 may generate the training data, which may include instructions for operating frequency of the imaging device 102, e.g., to facilitate focused ultrasound and/or to oscillate the bubble agents at their resonant frequencies. For example, the training device 101 may generate the instructions for the operating frequency of the imaging device 102 for ultrasound imaging of a target organ based on the scan depth of the ultrasound imaging realized from the first set of images and/or sensing data and/or based on the resonance frequency of the bubble agents.

Additionally or alternatively, during the training phase, the training device 101 may generate the training data, which may include beamforming information to generate the second set of images and/or sensing data, e.g., the focusing of the beams, corresponding to the predefined location, e.g., to deduce the location or position of a target organ. For example, the training device 101 may generate the beamforming information for the imaging device 102 for ultrasound imaging of the target organ based on the focus of the ultrasound imaging determined from the first set of images and/or sensing data.

Additionally or alternatively, during the training phase, the training device 101 may generate the training data, which may include the depth to generate the second set of images and/or sensing data, e.g., the depth of the beams for sensing, corresponding to the predefined location, e.g., corresponding to a target organ or tissue. For example, the training device 101 may generate the instructions for the imaging device 102 for ultrasound imaging of the target organ based on the scan depth of the ultrasound imaging determined from the first set of images and/or sensing data.

Additionally or alternatively, during the training phase, the training device 101 may generate the training data, which may include instructions regarding a detection of attachment anomalies, e.g., air bubbles, between the imaging device 102 and the skin, e.g., to generate notifications for the user to reattach or correctly attach the imaging device 102 upon detecting attachment anomalies between the imaging device 102 and the skin.

During the training phase, the training device 101 may train the imaging device 102 with the training data, which may include the generation of the images of a target organ, e.g., ultrasound images of the pancreas, such that the imaging device 102 may be able to recognize (e.g., intuitively) the correct (e.g., appropriate) position of a target organ, e.g., the pancreas, to carry out the sensing operation, e.g., focused ultrasound to excite the bubble agents, during the delivery phase.

For example, the imaging device 102 may transmit the second set of images and/or sensing data to the training device 101 wirelessly, e.g., via the link 103. Accordingly, the training device 101 may update the training data based on the second set of images and/or sensing data and may transmit the updated training data to the imaging device 102 wirelessly, e.g., via the link 103.

Alternatively, the training device 101 may wirelessly (e.g., via the link 103) control one or more parameters, e.g., the operating frequency, beamforming, depth of sensing, etc., of the imaging device 102. This may be useful for a recalibration of the imaging device 102, such as a change in beamforming and/or depth of sensing of the imaging device 102 due to the drift of the imaging device 102 with respect to the target organ caused by a movement, which may be determined based on the second set of images and/or sensing data. For example, the training device 101 may switch on or switch off the imaging device 102 wirelessly, e.g., via the link 103.

During the training phase, the imaging device 102 may generate the first set of images and/or sensing data under the supervision of the training device 101. For example, during the training phase, the training device 101 may include a high-resolution ultrasound device, and the imaging device 102 may generate ultrasound images of a target organ or raw/processed sensing data in relation to the target organ for ultrasound image reconstruction.

However, during the delivery phase, the imaging device 102 may generate the second set of images and/or sensing data (e.g., exclusively), such as ultrasound images of the target organ, which may be remotely with respect to the training device 101, such as based on the training carried out during the training phase.

During the training phase, the training device 101, based on the first set of images and/or sensing data, may train the imaging device 102 to recognize when the target organ is in a target location. Accordingly, during the delivery phase, the imaging device 102 may recognize the correct location of the target organ based on the second set of images and/or sensing data, and may proceed with the delivery phase, e.g., focused ultrasound to excite the bubble agents.

For example, during the delivery phase, the bubble agents may be excited, by the imaging device 102, without human intervention or without any supervision of the imaging device 102. For example, based on the training of the imaging device 102 during the training phase, the imaging device 102 may recognize the correct location of the target organ and may accordingly excite the bubble agents, e.g., by oscillating the bubble agents at their resonant frequency using ultrasound.

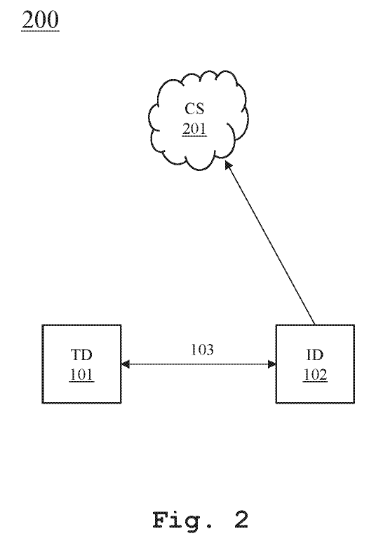

FIG. 2 shows a second example embodiment of an imaging system 200 according to the disclosure. The imaging system 200 differs from the imaging system 100 in that the imaging system 200 further includes a cloud storage (CS) 201. The imaging device 102 may be wirelessly coupled to the cloud storage 201.

Accordingly, the imaging device 102 may wirelessly transmit the first set of images and/or sensing data (e.g., during the training phase), the second set of images and/or sensing data (e.g., during the delivery phase), and the training data (e.g., during the training phase and/or delivery phase) to the cloud storage 201 for remote access and/or storage of the data.

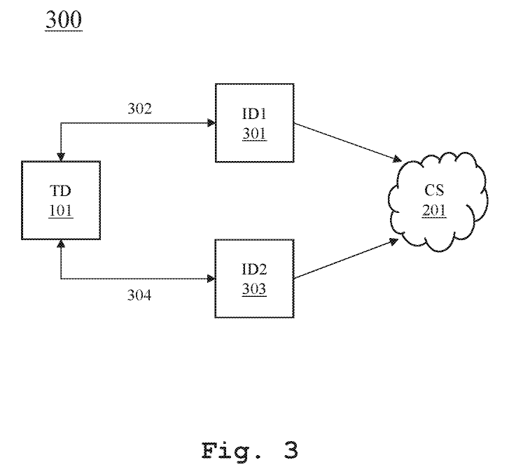

FIG. 3 shows a third example embodiment of an imaging system 300 according to the disclosure. The imaging system 300 may include the training device 101, a first imaging device 301, and a second imaging device 303, where each of the first imaging device 301 and the second imaging device 303 may correspond to the imaging device 102 of FIGS. 1 and 2. The imaging system 300 may include more than two imaging devices, as depicted herein.

The first imaging device 301 may be coupled to the training device 101 via a first link 302, which may be a wired link or a wireless link, and the second imaging device 303 may be coupled to the training device 101 via a second link 304, which may be a wired link or a wireless link.

For example, the first imaging device 301 may be attached to the skin of a user, e.g., a patient, at a first predefined location, e.g., corresponding to a target organ or tissue, and the second imaging device 303 may be attached to the skin of the user at a second predefined location.

For example, the first imaging device 301 may be attached to the skin of the user to generate abdominal ultrasound images, and the second imaging device 303 may be attached to the skin of the user to generate cardiac ultrasound images.

Alternatively, the first imaging device 301 may be attached to the skin of a first user at a first predefined location, and the second imaging device 303 may be attached to the skin of a second user at a second predefined location.

For example, the first imaging device 301 may be attached to the skin of the first user to generate abdominal or cardiac ultrasound images, and the second imaging device 303 may be attached to the skin of the second user to generate abdominal or cardiac ultrasound images.

A plurality of imaging devices may be trained by the training device 101, which may be used for targeted drug delivery at the respective plurality of locations, organs, or tissue, either simultaneously or sequentially.

During the training phase, the first imaging device 301, in combination with the training device 101, may generate images and/or sensing data corresponding to the first predefined location, and the training device 101 may generate training data for the first imaging device 301 corresponding to the first predefined location based on the generated images and/or sensing data.

Sequentially or simultaneously, during the training phase, the second imaging device 303, in combination with the training device 101, may generate images and/or sensing data corresponding to the second predefined location, and the training device 101 may generate training data for the second imaging device 303 corresponding to the second predefined location based on the generated images and/or sensing data.

During the delivery phase, the first imaging device 301 may (e.g., exclusively) generate (e.g., further) images and/or sensing data based on the training data corresponding to the first predefined location, such as from a location remote to the training device 101.

Sequentially or simultaneously, during the delivery phase, the second imaging device 303 may (e.g., exclusively) generate further images and/or sensing data based on the training data corresponding to the second predefined location, such as from a location remote to the training device 101.

The imaging system 300 may further include the cloud storage 201. Each of the first imaging device 301 and the second imaging device 303 may be wirelessly coupled to the cloud storage 201.

For example, the first imaging device 301 may wirelessly transmit the images and/or sensing data (e.g., during the training phase), the images and/or sensing data (e.g., during the delivery phase), and the training data (e.g., during the training phase and/or delivery phase) to the cloud storage 201.

Sequentially or simultaneously, the second imaging device 303 may wirelessly transmit the images and/or sensing data (e.g., during the training phase), the images and/or sensing data (e.g., during the delivery phase), and the training data (e.g., during the training phase and/or delivery phase) to the cloud storage 201.

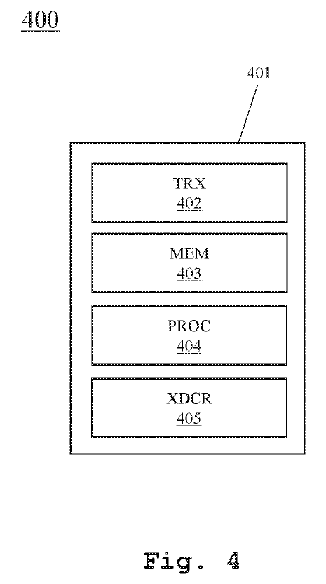

FIG. 4 shows an example embodiment of an imaging device 400 according to the second example embodiment of the disclosure. The imaging device 400 may correspond to the imaging devices 102, 301, 303 of FIGS. 1-3.

For example, the imaging device 400 may include a flexible substrate 401 that may be attached to the skin of a user, e.g., a patient. The flexible substrate 401 may be attached to the skin of the user mechanically, e.g., via strap or via a garment fitted onto the user, or via an adhesive.

For example, the flexible substrate 401 may be a relatively flexible material (e.g., able to elastically deflect over a defined distance without yielding) to reduce the risk of drift of the imaging device caused by a movement. The flexible substrate 401 may include or be composed of materials including ferro-electric polymers from polyvinylidene fluoride (PVDF) family of materials.

The imaging device 400 may further include a transceiver (TRX) 402, such as a wireless transceiver. The transceiver 402 may wirelessly transmit or receive data to or from the training device 101 of FIGS. 1-3 and/or the cloud storage 201 of FIGS. 2-3.

Additionally or optionally, the imaging device 400 may include a local storage or memory (MEM) 403, which may store the training data locally. The memory 403 may further store the images and/or sensing data generated by the imaging device 400.

Additionally or optionally, the imaging device 400 may include a processor (PROC) 404, which may process the training data to extract instructions and/or the raw data generated by the imaging device 400.

In addition, the imaging device 400 may include an imaging module (XDCR) 405, such as an ultrasound transducer, optionally an array of ultrasound transducers with embedded electronics to facilitate beamforming. The processor 404 may control the frequency and/or the beamforming of the imaging module 405 based on the generated images or sensing data and/or the training data.

The imaging device 400 may include a switch, such as a wireless switch, so that the training device 101 may switch on or off the imaging device 400 wirelessly.

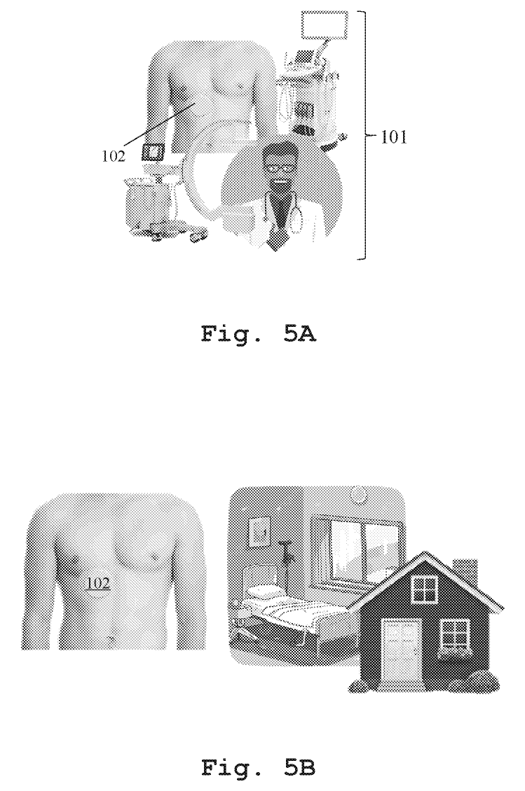

FIGS. 5A and 5B show an example operation of the imaging systems 100, 200, and 300. For example, FIG. 5A shows an example training phase of the imaging systems 100, 200, and 300, and FIG. 5B shows an example delivery phase of the imaging systems 100, 200, and 300.

For example, during the training phase, images and/or sensing data may be generated by one or more imaging devices, e.g., by the imaging device 102, such as in combination with the training device 101. Accordingly, the training device 101 may generate the training data, e.g., for the imaging device 102, based on the generated images and/or sensing data. During the training phase, the generation of the images or sensing data and the generation of the training data may be controlled by skilled personnel, e.g., a clinician.

For example, during the delivery phase, images and/or sensing data may be (e.g., exclusively) generated by one or more imaging devices, e.g., by the imaging device 102, that are independent of the training device 101 and based on the training data.

For example, the training data may be further updated based on the images and/or sensing data that are generated during the delivery phase, optionally under the control of the skilled personnel, e.g., via accessing the data from the cloud storage 201.

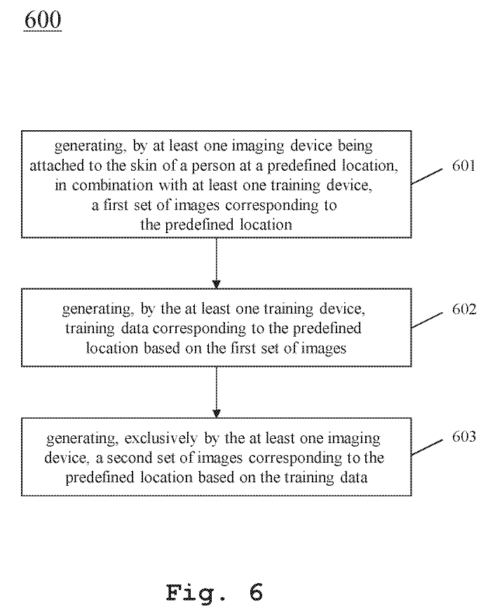

FIG. 6 shows an example embodiment of a method 600 according to the third example embodiment of the disclosure. In a first step 601, a first set of images corresponding to a predefined location are generated by at least one imaging device attached to the skin of a person at the predefined location in combination with at least one training device.

In a second step 602, training data corresponding to the predefined location are generated by the at least one training device based on the first set of images. In a third step 603, a second set of images corresponding to the predefined location are generated (e.g., exclusively) by the at least one imaging device based on the training data.

In the description as well as in the claims, the word “comprising” does not exclude other elements or steps and the indefinite article “a” or “an” does not exclude a plurality. A single element or other unit may fulfill the functions of several entities or items recited in the claims.

The term “and/or” used in the specification and the appended claims of the application refers to any combination and all possible combinations of one or more associated listed items, and includes these combinations.

The word “coupled” implies that the elements may be directly connected together or may be coupled through one or more intervening elements. Moreover, the disclosure with regard to any of the example embodiments is also relevant with regard to the other example embodiments of the disclosure.

Although the disclosure has been illustrated and described with respect to one or more implementations, equivalent alterations and modifications may occur to others skilled in the art upon the reading and understanding of the specification and the annexed drawings. In addition, while an example feature of the disclosure may have been disclosed with respect to (e.g., only) one of several implementations, the feature may be combined with one or more other features of the other implementations as may be useful for any given or example application.

While some example embodiments have been illustrated and described in detail in the appended drawings and the foregoing description, the illustration and description are to be considered illustrative and not restrictive. Other variations to the disclosed embodiments may be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. An imaging system for medical imaging comprising:
at least one training device; and
at least one imaging device coupled to the at least one training device, wherein the at least one imaging device is configured to attach to skin of a person at a predefined location,
wherein the at least one imaging device and the at least one training device are configured to generate a first set of images corresponding to the predefined location, wherein the at least one training device is configured to generate training data corresponding to the predefined location based on the first set of images, and
wherein the at least one imaging device is configured to generate a second set of images corresponding to the predefined location based on the training data.

2. The imaging system according to claim 1, wherein the at least one training device comprises at least one of an ultrasound imaging device, an x-ray imaging device, a computed tomography (CT) device, a magnetic resonance imaging (MRI) device, or a positron emission tomography (PET) device.

3. The imaging system according to claim 1, wherein the first set of images and the second set of images are ultrasound images.

4. The imaging system according to claim 3, wherein the first set of images and the second set of images are Doppler ultrasound images.

5. The imaging system according to claim 1, wherein the training data comprises instructions for an operating frequency of the at least one imaging device, focus of the second set of images corresponding to the predefined location, depth of the second set of images corresponding to the predefined location, or attachment anomalies between the at least one imaging device and the skin.

6. The imaging system according to claim 1, wherein the training data comprises parameters for a focused ultrasound based on the first set of images, and wherein the at least one imaging device is configured to perform the focused ultrasound at the predefined location during generation of the second set of images based on the training data.

7. The imaging system according to claim 1, wherein the at least one imaging device is configured to transmit the second set of images to the at least one training device.

8. The imaging system according to claim 7, wherein the at least one imaging device is configured to wirelessly transmit the second set of images to the at least one training device.

9. The imaging system according to claim 7, wherein the at least one training device is configured to update the training data based on the second set of images and further configured to wirelessly transmit the updated training data to the at least one imaging device.

10. The imaging system according to claim 1, wherein the at least one training device is configured to control at least one parameter of the at least one imaging device.

11. The imaging system according to claim 10, wherein the at least one training device is configured to wirelessly control the at least one parameter of the at least one imaging device.

12. The imaging system according to claim 10, wherein the at least one parameter comprises at least one of: an operating frequency of the at least one imaging device, a focus of the second set of images corresponding to the predefined location, or a depth of the second set of images corresponding to the predefined location.

13. The imaging system according to claim 1, wherein the imaging system comprises a storage configured to store at least one of the first set of images, the second set of images, or the training data.

14. The imaging system according to claim 13, wherein the storage is a cloud storage.

15. An imaging device for medical imaging comprising:
a flexible substrate configured to attach to the skin of a person at a predefined location;
a processor configured to process training data corresponding to the predefined location based on a first set of images generated in combination with at least one training device; and
at least one imaging module configured to generate a second set of images corresponding to the predefined location based on the training data, wherein the processor and the at least one imaging module are arranged on the flexible substrate.

16. The imaging device according to claim 15, wherein the flexible substrate is configured to attach to the skin of the person at the predefined location mechanically or via an adhesive.

17. The imaging device according to claim 15, wherein the at least one imaging module is a transducer.

18. The imaging device according to claim 17, wherein the at least one imaging module is an ultrasound transducer.

19. The imaging device according to claim 15, wherein the imaging device further comprises a transceiver configured to:
receive the training data from the at least one training device, wherein the training data is received wirelessly, or
transmit the second set of images to the at least one training device, wherein the second set of images is transmitted wirelessly.

20. An imaging method for medical imaging comprising:
generating, by at least one imaging device attached to the skin of a person at a predefined location and at least one training device, a first set of images corresponding to the predefined location;
generating, by the at least one training device, training data corresponding to the predefined location based on the first set of images; and
generating, by the at least one imaging device, a second set of images corresponding to the predefined location based on the training data.
